(19) United States
(12) Patent Application Publication
(10) Pub. No.: US 2021/0345270 A1
(43) Pub. Date: Nov. 4, 2021

(54) METHOD FOR SYNCHRONIZATION OF A MULTITUDE OF WEARABLE DEVICES

(71) Applicant: SMARTCARDIA SA, Lausanne (CH)

(72) Inventors: Srinivasan Murali, Lausanne (CH); Francisco Javier Rincon Vallejos, Renens (CH)

(21) Appl. No.: 17/269,469

(22) PCT Filed: Aug. 18, 2018
(86) PCT No.: PCT/IB2018/056260

§ 371 Date: Feb. 18, 2021

(51) Int. Cl. H04W 56/00; A61B 5/257; A61B 5/00; A61B 5/352; H04B 1/3827

(52) U.S. Cl. CPC H04W 56/0005; A61B 5/257; A61B 5/0006; H04W 4/80; A61B 5/0022; A61B 5/352; H04B 1/385; A61B 5/002

(57) ABSTRACT

In a method for synchronization of a multitude of wearable devices. Each wearable device is attached to a body (B) of a living organism. A software application running on a coordination device sends an application time information to each wearable device such as to allow the wearable devices to synchronize.

### TECHNICAL FIELD

The disclosure relates to a method for synchronization of a multitude of wearable devices.

### BACKGROUND ART

For accurate monitoring of several conditions of living organisms, such as for example Atrial Fibrillation (AF), a multiple lead electrocardiogram (abbreviated as ECG) monitoring is needed, so that if there is noise in one of the leads, another lead can be used. In traditional systems, the different lead wires are connected to different parts of the body of the living organism on one end, and the wires are connected to a single electronic unit on their other ends. Thus, all the different ECG lead signals are time synchronized or can be easily synchronized because they are all interconnected with each other and with the electronic units by means of cables.

For other health conditions, multiple bio-signals (such as one or more lead ECG, pulse from finger) are measured and monitored. In traditional systems, the different measurement units are connected to an electronic unit by means of cables, and the time synchronization between them can also be performed easily by the electronic unit.

Recently, as an alternative to cable-based ECG and bio-sensing systems, wearable devices have been presented. Such wearable devices can for example take the form of wearable patches that comprise an electronic circuitry comprising for example a processor, one or more sensors and a transmitter. Furthermore, such wearable patches typically comprise an adhesive surface by means of which they can be attached to a body. When using such wearable devices and in particular wearable patches, given the small size of such devices, it is possible to use multiple wearable devices and/or patches at different parts of the body to obtain multi lead ECG signals and measure other bio-signals, such as the pulse, blood pressure, phonocardiogram or impedance cardiogram. A single patch at a location could also measure more than one bio-signal simultaneously.

However, because the wearable devices are not interconnected by means of cables, it is not easy to properly time synchronize the different wearable devices. Such a time synchronization is, however, important in order to accurately align the data (such as for example ECG signals) obtained from the different patches temporally (that is, for example on a common time axis).

Furthermore, time differences of body signals measured at different parts of the body by means of wearable devices such as the previously mentioned wearable patches can in principle be used to acquire health information concerning the body. However, for such health information to be accurate, the different wearable devices should be time synchronized as precisely as possible.

### SUMMARY

It is one object of the disclosure, per an embodiment, to solve or to at least diminish the above-mentioned disadvantages. In particular, it is an object of the disclosure, per an embodiment, to find ways to enable and/or to improve the time synchronization of wearable devices, for example wearable patches, which are being employed for measuring body signals.

This problem is solved, according to one embodiment, by a method for synchronization of a multitude of wearable devices, wherein each wearable device is attached to a body of a living organism, wherein a software application running on a coordination device sends an application time information to each wearable device such as to allow the wearable devices to synchronize.

In the context of this application, the term “synchronization” refers to a time-wise synchronization. The expression “wearable device” is to be understood as any electronic device that is configured to be at least temporarily attached to a body, for example a wearable patch as previously described or a watch or smartwatch, or a wrist band, a chest band or a collar. The term “multitude” is to be understood in the sense of “at least two”. A “body of a living organism” is typically a human body or an animal body. A “software application” is to be understood as any kind of computer program, for example a software tool running on a desktop computer or laptop, or yet an application running on a smartphone, mobile phone, tablet, smartwatch or any other portable electronic device, or yet a cloud-based application. The term “coordination device” is to be understood such that it relates to any kind of electronic device that is able to run a software application in the sense of the present disclosure. It is not excluded that the coordination device is at the same time a wearable device in the sense of the application. For example, a smartwatch can theoretically at the same time be part of the multitude of wearable devices and serve as coordination device. The term “application time information” typically refers to an absolute time, such as “12:30:44”, i.e. 12 h 30 m 44 s, but can in principle also refer to a relative time, for example the current value of a counter which has been started at a particular moment in time.

The fact that there is one coordination device with a software application running on it and that this software application sends an application time information to all wearable devices may be an effective way to enable synchronization of the wearable devices.

In certain embodiments, at least one of the wearable devices is a wearable patch for monitoring at least one body signal, in particular a pulse signal and/or an ECG signal, of the body. In the context of this application, the term “wearable patch” refers to a device that comprises an electronic circuitry, wherein the electronic circuitry typically comprises a processor and/or a sensor, typically a pulse sensor, and/or a transmitter. Furthermore, such wearable patches may comprise an adhesive surface by means of which they can be attached to a body. The use of wearable patches can be advantageous because they are typically especially designed for measuring body signals such as ECG signals or pulse signals. In addition to that, such patches are typically comparably small and/or flat and can be attached to various parts of the body, e.g. on chest, stomach, arm or leg of human being or animal. However, it is not mandatory to use wearable patches: it is also possible to use for example a combination of a smartwatch and a chest band as wearable devices. In certain embodiments, all wearable devices are wearable patches. This may have the advantage that synchronization can be particularly straightforward because there is only one device type. In other typical embodiments, one wearable device is a smartwatch and the other wearable devices are wearable patches.

In certain embodiments, the coordination device is a mobile phone and/or a tablet computer and/or a laptop, and/or a computer and/or a Bluetooth hub and/or a router and/or any type of hardware device, preferably any type of portable electronic device. The use of such devices as coordination device may have the advantage that these devices are widely available and can all easily be configured to facilitate the execution of the software application. In an embodiment, the coordination device is a system with distributed components, for example a combination of a smartwatch, a smartphone and a remote computer. In an embodiment, the coordination device comprises a smartwatch or is a smartwatch. In an embodiment, the coordination device is at the same time a wearable device in the sense of this disclosure.

In certain embodiments, the application time information is sent to the wearable devices via a wireless network, preferably, per an embodiment, using a wireless protocol such as Bluetooth and/or Zigbee and/or WiFi and/or GSM. The use of such networks may be advantageous for example because they are standard networks that are widely available and reliable and have well-defined characteristics. However, it is in principle also possible that the time information is sent through point-to-point connections and/or using a custom protocol.

In certain embodiments, the application time information corresponds to an internal time of the coordination device, wherein the internal time is preferably, per an embodiment, a network time, in particular a time provided by a mobile carrier network, or a time set by the user. Choosing one of these times as application time information may be advantageous, because these times are typically easily available and easy to understand. However, the application time information can in principle also correspond to a more abstract value, such as the value of an internal counter of the coordination device.

In certain embodiments, each wearable device stores the application time information, preferably, per an embodiment, in a wearable device processor comprised in each wearable device, and starts a counter for maintaining a wearable device time in line with the application time information, preferably, per an embodiment, in line with the internal time of the coordination device. In the case where a wearable device is actually a wearable patch, the wearable device processor is referred to as patch processor and the wearable device time is referred to as patch time. Starting an internal counter in each device based on the application time information received from the coordination device may be advantageous because once the application time information has been received by the different wearable devices, each device can in principle maintain its proper wearable device time, which is in line—and thus synchronized—with wearable device times of any of the other wearable devices. Like this, no further interaction with the coordination device is in principle necessary anymore, and it is for example possible to at least temporarily switch of the coordination device or cut off a connection between the multitude of wearable devices and the coordination device. Furthermore, it is possible to then change the coordination device. However, counters in the wearable devices are not absolutely mandatory. It is for example also possible for the coordination device to periodically send application time information to the wearable devices.

In certain embodiments, in each particular wearable device, the counter of that particular wearable device uses a sampling rate of at least one sensor comprised in that particular wearable device for counting, wherein the sensor is preferably, per an embodiment, a pulse sensor. As a matter of fact, sensors often comprise oscillating crystals for maintaining sampling rates of the sensor, and the sampling rates created by these oscillating crystals can therefore advantageously be used as counters of the wearable devices. Put differently, in certain embodiments, an oscillating crystal comprised in a sensor of the wearable device forms part or is the counter. This may have the advantage of reducing the number of components in the wearable devices. It is of course also possible that each particular wearable device comprises a separate counter, for example one that comprises a distinct oscillating crystal, or that only some or one of the wearable devices use a sampling rate of at least one sensor comprised in that particular wearable device for counting.

In certain embodiments, the multitude of wearable devices comprises at least two, preferably at least three, more preferably at least five wearable devices, wherein the wearable devices are preferably essentially identical. The expression “essentially identical” is to be understood such that the wearable devices are in principle able to all fulfill the same functions but that it is not excluded that they for example slightly differ in size or form. In certain embodiments, all wearable devices are identical. In other embodiments, some of the wearable devices are identical and others are not. For example, it is possible that the multitude of wearable devices comprises two identical wearable patches placed at different parts of a body, e.g. on the chest and on the stomach, and a smartwatch placed at a wrist of the body. Essentially identical or identical wearable devices can simplify the synchronization because they are likely to function in a highly similar manner for example as much as individual processing speeds are concerned. It is, however, also possible that all wearable devices are different and/or that another amount of wearable devices, for example four wearable devices, is used.

In certain embodiments, the method comprises a first calibration phase, wherein the first calibration phase comprises the steps:
each wearable device measures a first characteristic point of a body signal, wherein the body signal is typically an ECG signal of the living organism, wherein the characteristic point is typically an R-peak of a QRS complex of the ECG signal;
each wearable device determines time-stamps of subsequent characteristic points, typically being of a same type as the first characteristic point, based on its particular counter;
each wearable device sends the time-stamps to the coordination device.

It may be mandatory, per an embodiment, that each wearable device participates in the first calibration phase. For example, it is possible that one or more wearable devices are located too remotely on a body (for example on the arm or leg) and that a body signal such as an ECG cannot be measured. Therefore, in certain embodiments, only those wearable devices which are actually able to receive an ECG signal at the location of the body where they are placed participate in the first calibration phase. In this case, only those wearable devices which participate in the first calibration phase measure a first characteristic point of a body signal and carry out the subsequent steps of the first calibration phase. Furthermore, a body signal in the sense of the present disclosure is not necessarily a direct body signal. In certain embodiments the body signal is, at least for one or more wearable devices, an indirect body signal, for example an electrical impulse sent out by a wearable device able to receive an ECG signal. This concept is explained in more detail further below.

A time-stamp in the sense of the present disclosure can be an absolute time, for example 11:58:34 or 17:44:12, or yet a relative time, like for example 1.24 seconds. The advantage of a calibration phase, per an embodiment, during which each wearable device sends time-stamps corresponding to characteristic points of a body signal of the body to which the wearable devices are attached to the coordination device is the following: in this way, the coordination device can become aware of synchronization problems between the different wearable devices, for example because it can become aware of the fact that the time-stamps for characteristic points of a body signal—such as the ECG—do not match for all wearable devices and that at least some wearable devices are thus not properly synchronized. In fact, an ECG signal typically propagates so quickly through the body that wearable devices which are configured to detect this ECG signal but which are located at different locations of the body should all receive the ECG signal at essentially the same time (i.e. with a negligible time shift in the range of a few microseconds), and thus all time-stamps should theoretically be the same for all wearable devices if the devices are properly synchronized. If they are not, this hints towards a synchronization problem, and the coordination device can in such a case take appropriate measures, for example re-initiating a synchronization. The advantage of the transmission of several time-stamps, per an embodiment, is that it makes it easier for the coordination device to for example detect a systematic “drag behind” of one or more wearable devices. However, in theory one time-stamp per wearable device can be enough for the coordination device to detect a synchronization problem that needs connection.

At this point, one reason why good synchronization is desirable per some embodiments when using various wearable devices for measuring body functions shall be pointed out: one possible way to use the measured body function data is to display the body functions on a screen, for example a screen of the coordination device, with a common time axis. If the synchronization between the wearable devices is not good, then the representation on the screen is improper because the data from the different wearable devices is misaligned on the time axis.

In certain embodiments, the first calibration phase is followed by a second calibration phase, wherein the second calibration phase comprises:
a time offset calculation step during which the coordination device calculates a particular time offset value for at least one of the wearable devices, preferably for each wearable device, based on the time-stamps received from the wearable devices,
an offset transmission step during which the coordination device transmits at least to each of those wearable devices for which the particular time offset value does not equal “0” and/or to each of those wearable devices for which a time offset value has been calculated its particular time offset value;
a time adjustment step during which each wearable device that received its particular time offset value from the coordination device uses this particular time offset value to offset its particular wearable device time.

The inventors have found that it can sometimes be tricky to reach good synchronization because the transmission of the application time information from the coordination device, the processing time of this application time information in the different wearable devices as well as the time until the wearable devices start their respective counters based on the application time information are not necessarily equal across the multitude of wearable devices. With the proposed second calibration step, each wearable device that is not properly synchronized receives a customized time offset value and can thus adjust its particular wearable device time based on this offset value. Like this, the overall synchronization of the multitude of wearable devices can be improved. However, such a second calibration step may not be absolutely mandatory. It would for example also be possible that the coordination device simply re-initiates the synchronization process as often as necessary until any time shifts between the different wearable devices are in an acceptable range. Furthermore, it would also be possible to not at all tell the wearable devices about their possible respective mis-synchronization and to only keep track of them in the coordination device. However, in such a case, it would be more difficult to switch the coordination device subsequently. In other words: It will be easier to switch the coordination device later on if the wearable device times of all wearable devices are properly synchronized.

In certain embodiments, during the time offset calculation step, the coordination device calculates a mean time difference based on the time stamps for at least one of the wearable devices and saves this mean time difference as the particular time offset value. The calculation of a mean time difference may be advantageous because it can equal out variations and can therefore lead to more reliable time offset values. The mean time difference can correspond to an arithmetic mean or to a median.

In certain embodiments, the time offset calculation step comprises the sub-steps:
the coordination device preferably chooses one wearable device as reference wearable device;
the coordination device calculates time differences between the time-stamps of the reference wearable device and corresponding time-stamps of each other wearable device such as to obtain a multitude of time differences for each pair of the reference wearable device and any of the other wearable devices;
for each pair of the reference wearable device and any of the other wearable devices, the coordination device calculates a mean time difference based on the respective multitude of time differences;
for each other wearable device, the coordination device saves the determined mean time difference as the particular time offset value;

and the offset transmission step comprises the sub-step:
the coordination device transmits to each of the other wearable devices its particular time offset value.

This particular configuration of the time offset calculation step may have the advantage to lead to comparably precise time offset values for all wearable devices and therefore to a comparably precise synchronization result.

In certain embodiments, the wearable devices only transmit information to the coordination device when the signal quality is good enough. In this context, the expression “information” relates in particular to time-stamps and/or waveforms and/or body signals. The advantage of this, per an embodiment, is for example that the synchronization method is optimized because the coordination device is not bothered with noisy information that could have a negative impact on the calculations carried out by the coordination device, for example the calculations of the time offset values. In certain embodiments, a wearable device that is experiencing noise sends a noise flag to the coordination device when noise is present and/or sends a noise start flag to the coordination device when noise starts and a noise end flag when noise has ended. In certain embodiments, autocorrelation is used to identify appropriate time offset values in cases where noise is present. In certain embodiments, autocorrelation is at least partly performed by shifting body function signals from at least two wearable devices against each other on a time axis until an optimal correlation is found.

In certain embodiments, the characteristic point is a peak of pulse or a foot of pulse and/or not all wearable devices use the same type of characteristic point, wherein each wearable device preferably uses a different type of characteristic point. The use of pulse wave characteristics as characteristic points may have the advantage of making it possible to enable synchronization also in cases when ECG measurements are not available. The combination of different types of characteristic points, for example R-peaks and peaks of pulse may have the advantage of allowing synchronization also in cases where some but not all wearable devices can measure ECG signals. In embodiments where different types of characteristic points are combined, a constant offset is typically applied to the time-stamps relating to one or more of the different characteristic points.

In certain embodiments, a characteristic point, in particular an R-peak, is considered to be noisy if an RMS of an accelerometer value around that particular R-peak exceeds a certain threshold. This may be advantageous because the wearable devices often comprise accelerometers in any case, because the RMS calculation is straightforward and because the higher the RMS of an accelerometer value is the more noise is typically comprised in the body signals, in particular the ECG, measured by the particular wearable device. RMS is an abbreviation for “Root Mean Square”.

In certain embodiments, at least one wearable device periodically sends a distinct shape electrical impulse, preferably a square shape impulse, through the body to the other wearable devices so that the other wearable devices can use that distinct shape electrical impulse for synchronization. This may have for example the advantage that wearable devices which are not able to measure an ECG signal—for example because they are located too far away from the heart, for example at a wrist—can use this distinct shape electrical impulse as characteristic point and can establish their time-stamps to be sent to the coordination device based on these electrical impulses. In certain embodiments, the distinct shape electrical impulse is in line with an ECG signal, typically with an R-peak of the ECG signal. In certain embodiments, the distinct shape electrical impulse is used as indirect body signal by at least some of the wearable devices during the first calibration phase and/or during the second calibration phase.

In certain embodiments, the distinct shape electrical impulse has an amplitude that is higher than a typical amplitude of an ECG signal, wherein the amplitude of the distinct shape electrical impulse is preferably higher than 10 mV, more preferably higher than 15 mV, most preferably higher than 20 mV.

In certain embodiments, the distinct shape electrical impulse is generated by at least one wearable device when this at least one wearable device measures a characteristic point on a particular body signal, such as the R-wave of the ECG signal or the foot and/or peak of the pulse signal. This may have the advantage of allowing a good periodicity of the distinct shape electrical impulse.

In certain embodiments, a moving time window and/or an instantaneous frequency waveform is used for synchronization in heavy noise.

In certain embodiments, at least one timing value is calculated once all patches are synchronized. In certain embodiments, the timing value comprises a Pulse Arrival Time (PAT) and/or a Pre-Ejection Period (PEP) and/or a Pulse Wave Velocity (PWV).

### BRIEF DESCRIPTION OF THE FIGURES

In the following, the disclosure is described in detail by means of drawings, wherein show

FIG. 1: a schematic view of a part of a human body to which two wearable devices are attached (one wearable device attached to chest, one wearable device attached to arm),

FIG. 2: a schematic diagram of an ECG signal and an associated pulse signal,

FIG. 3: a schematic view of a part of a human body to which two wearable devices are attached (both wearable devices attached to arm),

FIG. 4: a schematic diagram of an ECG signal and an associated electrical impulse signal.

### DETAILED DESCRIPTION

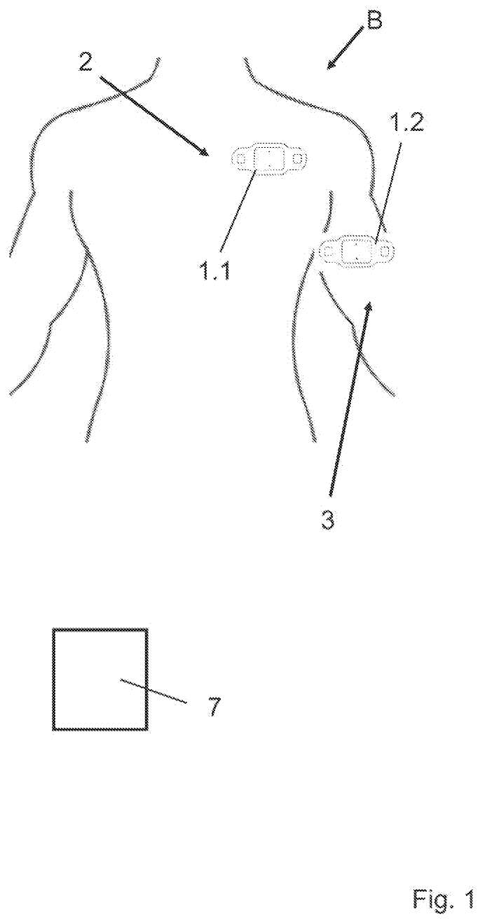

FIG. 1 shows a part of a human body B. Two wearable devices 1.1, 1.2 are attached to the human body B. The wearable devices 1.1, 1.2 have the form of wearable patches, which are attached to the human body B by means of an adhesive surface. Each of the wearable patches 1.1, 1.2 comprises an electronic circuitry, wherein the electronic circuitry comprises a processor and a multitude of sensors, especially a pulse sensor, an ECG sensor and an accelerometer, and wherein the electronic circuitry comprises a receiver and a transmitter. Furthermore, each wearable patch 1.1, 1.2 comprises an adhesive surface by means of which they are attached to the body B.

Wearable patch 1.1 is attached to a chest 2 of the human body B and wearable patch 1.2 is attached to an upper arm 3 of the human body B.

The two wearable patches 1.1, 1.2 are time synchronized by means of a coordination device 7. The coordination device 7, which can for example be a mobile phone, has a software application running on it. This software application enables and/or facilitates connection between the coordination device 7 and the wearable patches 1.1, 1.2, transmission and reception of data to/from the wearable devices 1.1, 1.2 as well as visualization of this data, for example data relating to body functions of the human body B sampled by each wearable patch 1.1, 1.2.

When a user places a wearable patch 1.1, 1.2 on the body B (for example on a chest 2, as shown for wearable patch 1.21 in FIG. 1) and connects the wearable patch 1.1, 1.2 to the software application running on the coordination device 7, the software application transmits an application time information to this particular wearable patch 1.1, 1.2. In the case where the coordination device 7 is a mobile phone, this application time information is typically the current time of the mobile phone.

In some embodiments, the connection protocol used for the communication between the coordination device 7 and the wearable patches 1.1, 1.2 is Bluetooth or Wireless or any standard protocol. In some embodiments, the current time of the mobile phone is a network time (such as provided by the mobile carrier network) or a time set by the user.

In the wearable patch 1.1, 1.2, a processor stores the application time information and starts a counter for maintaining a wearable device time. This is preferably done, per an embodiment, by using a sampling rate of one of the sensors of the wearable patch 1.1, 1.2. For example, if the pulse sensor of a particular wearable patch 1.1, 1.2 is sampled at 250 Hz, then after receiving 250 samples from the pulse sensor, the wearable device time on the processor of that wearable patch 1.1, 1.2 is updated by 1 second. To have a finer granularity, for each sample received from the sensor, the timer can be updated by 1/sampling rate value. For example, where the sampling rate of a sensor is 250 Hz, for each sample received from the sensor, the wearable device time is updated by 4 milliseconds.

When a second wearable patch 1.1, 1.2 is placed on the human body B (for example on an arm 3, as shown for wearable patch 1.2 in FIG. 1), it is also connected to the same software application running on the coordination device 7. Just as the first wearable patch 1.1, 1.2, this second wearable patch 1.1, 1.2 also receives the application time information from the software application, stores the application time information in its processor and starts a counter for maintaining a wearable device time. The same typically happens for any further wearable devices placed on the human body B (even if FIG. 1 only shows two wearable patches 1.1, 1.2). Thus, for each of the different wearable patches 1.1, 1.2 (and any additional wearable devices) placed on different locations of the human body B, a reference time based on the current time of the coordination device 7 is set.

In other embodiments, the coordination device 7 comprises or is a Bluetooth hub, a computer or a router.

In other embodiments, another protocol to connect the wearable devices 1.1, 1.2 to the coordination device 7 is used, for example any another wireless protocol, such as Zigbee, WiFi or GSM. It is also possible to combine different protocols.

Due to the nature of the Bluetooth protocol or any other underlying protocol used for connecting the coordination device 7 to the wearable devices, a slight delay between transmission of the application time information from the coordination device 7 to the wearable devices on one hand and the actual receipt and storage of the application time information on the processor of the wearable devices on the other hand can occur. This delay can be in the order of milliseconds to few seconds, depending on the mechanism of the underlying protocol and distance between the coordination device 7 and the wearable devices. This delay is therefore not necessarily constant for all wearable devices.

In an embodiment, the method for synchronization of a multitude of wearable devices therefore comprises a first calibration phase for improving the time synchronization of the different wearable devices placed on a body. During this first calibration phase, each wearable device measures a characteristic point of the ECG signal, such as the R peak of the QRS complex. Each wearable device then transmits time-stamps of the subsequent R peaks to the coordination device.

In an embodiment, the method for synchronization of a multitude of wearable devices furthermore comprises a second calibration phase for further improving the time synchronization of the different wearable devices placed on a body. During this second calibration phase, the differences between the time stamps of the R peaks from the different wearable devices are calculated in the coordination device. The mean or median value of multiple differences of the time stamps can be calculated. In the case shown in FIG. 1 (i.e. where there are two wearable devices 1.1, 1.2), the coordination device 7 chooses the time stamp from one of the wearable devices 1.1, 1.2 to be reference and transmits the time difference to the other wearable device 1.1, 1.2. The other wearable device 1.1, 1.2 adjusts its particular wearable device time based on the difference. After this step, the second calibration phase is completed. The body signals from the two wearable devices 1.1, 1.2 can now be displayed on the same screen with the same time reference. The second calibration phase can of course also be carried out with more than two wearable devices.

During the first calibration phase and the second calibration phase, the ECG is thus used for calibration. The ECG is an electrical signal that propagates quickly (in microseconds) across the body, as the body is a good conductor of electricity. The pulse wave, on the other hand, could take hundreds of milliseconds to move from the heart to a peripheral part of the body. Thus, ECG acquired from distinct points of the body can be used for improving synchronization of the timing information of the corresponding wearable devices.

In some embodiments, the time synchronization is performed only when the data acquisition of the wearable devices is of good quality. For example, for each patch, the time differences between two subsequent R peaks (the RR interval) are computed on the wearable device when the signal quality is good, and are transmitted to the coordination device with the time stamp of the R-peak and/or the second R-peak. In case of a bad signal quality, the R peaks are discarded from computation. The values of RR intervals from the different patches are stored on the coordination device. After a predefined time, the autocorrelation of the RR intervals from the different wearable devices at different time lags are performed. The time lag that provides the best correlation is then transmitted to one of the wearable devices (or several of the wearable devices, in case there are more than two wearable devices) for time synchronization.

It is also possible to perform the described synchronization method using pulse signals, with peak or foot of the pulse used as a characteristic point (similar to the R peak of ECG). The same synchronization mechanism can also be applied between the ECG on one patch and pulse on the other patch. However, as the pulse signal usually takes some time to reach a point in the body, a constant offset needs to be added to the time difference in such a case.

In some embodiments, the RMS (Root Mean Square) of the accelerometer value delivered by an accelerometer of a particular wearable device is checked around the R peak, and if the RMS exceeds a threshold, the R-peak value and timing information are not transmitted by this particular wearable device to the coordination device and the signal is marked as noisy.

In another aspect of an embodiment of the disclosure, once all wearable devices are synchronized, certain timing values between the body signals measured by the different wearable devices are computed.

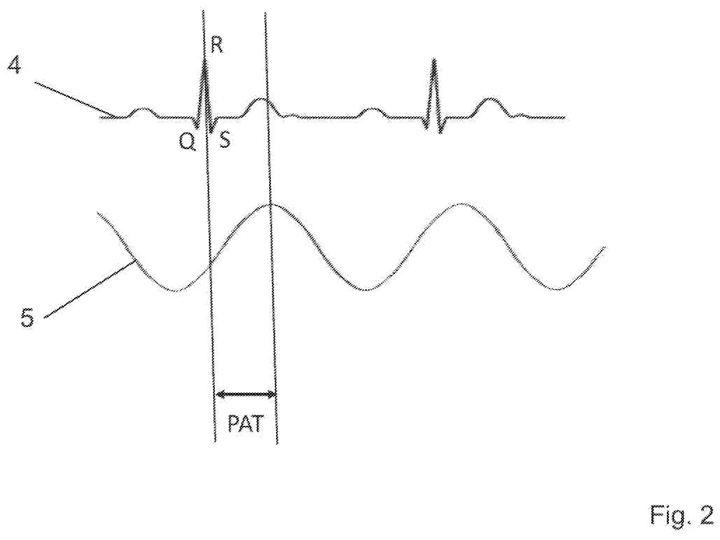

For example, the Pulse Arrival Time (PAT) is such a timing value. An example of an ECG signal 4 and pulse signal 5 with the PAT marked is shown in FIG. 2. The PAT is computed as the time interval between the ECG R-peak and a characteristic point on the pulse signal (such as the pulse peak or pulse foot—in FIG. 2, it is shown for the pulse peak). The pulse arrival time has two components, Pre-Ejection Period (PEP) and the time the pulse wave takes from the blood flow out of aorta to the point at which the patch is placed. The distance between the blood flow out of aorta and the point where the patch is placed is represented by k (in meters). The PAT is computed using the following formula:

PAT=PEP+k/PWV,

wherein PWV is the pulse wave velocity.

When two wearable patches are placed on the same user at different points, the PAT at the two points can be used to compute PWV and PEP values. Let k1 and k2 be the distances from the heart to the point where two patches are placed. Then the respective PAT can be computed as follows:

PAT1=PEP+k1/PWV

PAT2=PEP+k2/PWV

The PWV is computed using the following equation:

PWV=(k1−k2)/(PAT1−PAT2)

Once the PWV is computed, the PEP value can be computed. The precise determination of the timing values PAT, PEP and PWV may be advantageous because these timing values can give valuable insights into possible malfunctions of the body. It may be important to understand that the better the wearable devices placed on the body which are used to calculate these timing values are synchronized, the more precisely the timing values can be determined and the more precisely body malfunctions can be detected.

One of the challenges of the method for computing the timing values described above is that the distances k1, k2 etc. of where the patches are placed from the heart need to be computed. This can be challenging with differing geometries of the arteries for each user.

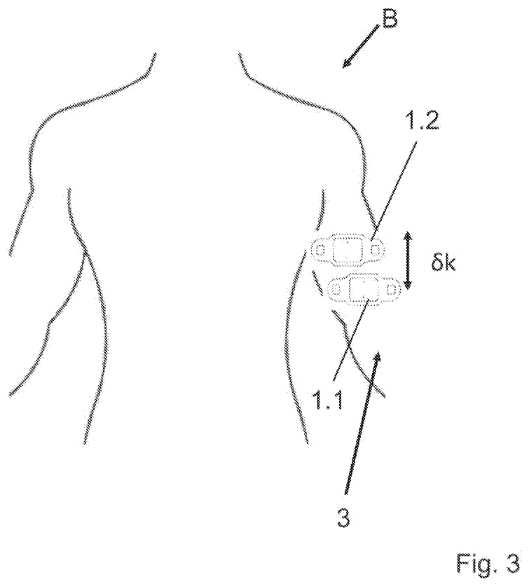

In another aspect of an embodiment of the disclosure, in order to yet improve the determination of the timing values, two wearable patches 1.1, 1.2 are placed at a predetermined distance bk from each other. For example, the patches could be placed next to each other, horizontally stacked on an upper arm 3 of a human body B, as shown in FIGS. 3.

In this case, the PWV is computed as follows:

PWV=bk/(PAT1−PAT2),

where bk is the distance between the wearable patches 1.1, 1.2.

In another aspect of an embodiment of the disclosure, two wearable patches are put on two sides of a body of a living organism. For example, one wearable patch is put on each of the upper arms of a human body, and both wearable patches are placed at the same height. In an experimental phase, the PWV is calculated by another method, such as placing two wearable patches at a known location or using a different technique, such as the arterial tonometer. The height of the user is also taken as input for the method. The differences in the pulse arrival times (PAT1 and PAT2) are computed from the patches on both sides, along with the height of the user and are correlated with the PWV computed by the other method. This correlation is further used to build a model of (PAT1-PAT2) and height with measured PWV. In actual usage, this model is used to measure PWV, for a given (PAT 1-PAT2) and a given height of the user.

In yet another embodiment of the disclosure, two wearable patches are placed on chest and abdomen and synchronization according to the disclosure is performed using the ECG R-peaks or pulse wave. The pulse signals of the two wearable patches are further aligned and filtered by a low pass filter with the respiration band. The two signals are added, and a new signal is created. This signal is used as respiration wave of the user.

Even though the determination of the timing values is explained for wearable patches in the above, the determination of the timing values is of course in principle possible with any case of wearable device.

In some embodiments, each wearable device, in particular each wearable patch comprises an additional circuitry for inducing an electric current, preferably a low amplitude current, into the body to which the wearable devices are attached at a predetermined rate, preferably mimicking an electric potential of the Sino Arterial node of the heart. In certain embodiments, the shape of the injected current, which is also referred to as signal, pulse, impulse or electrical impulse signal, is fixed to a value distinct from that of the ECG QRS complex (for example a square wave instead of a triangular QRS complex). When a wearable device injects such a signal into the body of the user of the wearable device, other wearable devices attached to the same body typically receive the signal in the same channel as they would use for sampling the ECG signal. Since the shape of the induced signal is distinct, it can be separated from the ECG signal of the heart.

In an embodiment, the wearable device injecting the electrical impulse signal does so every time it receives an R-peak from the ECG signal. This electrical impulse signal is received quasi-instantaneously at the other wearable devices attached to the same body (due to low resistance of the human body).

The wearable devices which receive the electrical impulse signal can either discard it (typically if they receive the ECG signal themselves) or can use it as an indirect indication of the R-peak of the ECG signal. In particular, in certain embodiments, wearable devices which are placed at parts of the body where they cannot measure the ECG signal (e.g. on an arm or a leg) receive the electrical impulse signal and use it as indirect body signal for use in the first calibration phase.

In certain embodiments, the injected electrical impulse signal is set to have a completely different amplitude (for example much higher) than the ECG signal of the heart, so that the receiving wearable devices can easily differentiate the injected electrical impulse signal from the heart ECG signal.

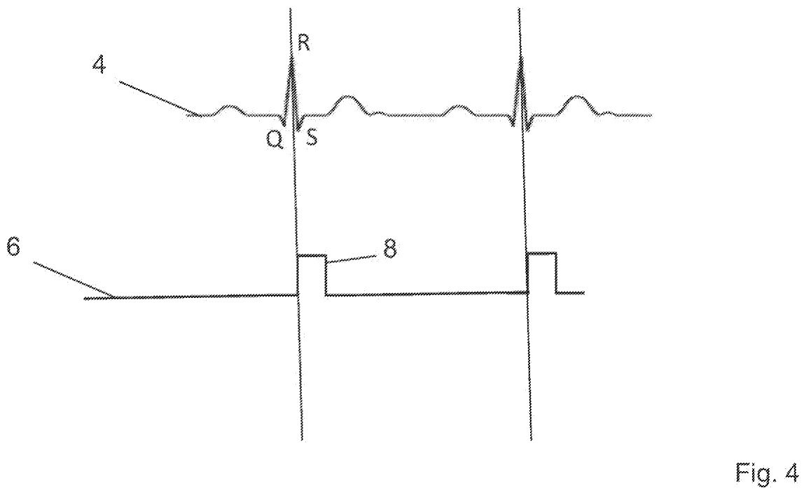

FIG. 4 shows an example of an ECG signal 4 and an associated electrical impulse signal 6. Typically, a wearable device which monitors a body's ECG signal 4 creates the electrical impulse signal 6 by forming a rectangular electrical impulse 8 at every time it determines an R-peak in the ECG signal 4. This wearable device injects the electrical impulse signal 6 into the body and thereby enables other wearable devices to make use of the electrical impulse signal 6, for example by using it as an indirect body signal during the first calibration phase.

For example, in the situation shown in FIG. 1, one could imagine that the wearable patch 1.1 attached to the chest 2 is able to measure the ECG signal of the human body B but that the wearable patch 1.2 attached to the arm 3 is not able to receive the ECG signal of the human body B. If the wearable patch 1.1 injects the electrical impulse signal 6 into the human body B, then the wearable patch 1.2 can use the electrical impulse signal 6 as (indirect) body signal and thereby participate in the first calibration phase and typically also in the second calibration phase. This may have the advantage to achieve better synchronization results because also remotely located wearable devices can participate in the first and second calibration phases.

In certain embodiments, particular steps are taken for synchronization in heavy noise. For example, the ECG and pulse signals can be highly corrupted by movement of the user or other noise sources, such as 50 Flz/60 Hz power supply noise, electromagnetic interference, etc. In some embodiments, an adaptive filter is used to carry out the timing synchronization under heavy noise. For this, the ECG signal for a pre-determined time window is stored on a wearable device, e.g. a wearable patch. The time window is moved in a sliding fashion, so that computations can be performed in a finer granularity of time window. In some embodiments, the ECG signal for eight seconds is stored on the wearable device for analysis, and the sliding window has a width of one second, so each second computations are performed using the preceding eight seconds of the ECG signal. The noise on the ECG signal is adaptive cancelled using the accelerometer data of the wearable device in question in the same interval.

In certain embodiments, after the noise cancellation, an adaptive band-pass filter is used to track the instantaneous frequency component, which is associated with the heart rate of the user. The instantaneous frequency waveform over a predetermined time interval is then sent to the coordination device. In some embodiments, the instantaneous frequency is computed every one second on an ECG data of eight seconds, in a sliding window fashion. The frequency computed every second for a 60-second duration is then sent to the coordination device.

In certain embodiments, the auto correlation of the frequency signals from two wearable devices at different time stamps is performed on the coordination device, and the time lag that maximizes the correlation is chosen as the time difference between the time stamps on the wearable devices and used for synchronization.

In the above described method, if the signal is noisy, multiple instantaneous frequencies could appear over each time window. In such a case, in one embodiment of the disclosure, the maximum frequency across the different ones is chosen for the window. In another aspect of an embodiment of the disclosure, the frequency component that is closest to the frequency component chosen in the previous window (or over several previous windows) is chosen as the value.

The disclosure is applicable to wearable devices, electronic skin sensors for patient monitoring at homes, hospitals, as well as healthy subject monitoring. In fact, the method of synchronization can be used for synchronizing watches or other devices with one another on the same user, using the user's bio signals.

The invention is not limited to the embodiments described here. The scope of protection is defined by the claims.

Furthermore, the following claims are hereby incorporated into the Description of Preferred Embodiments, where each claim may stand on its own as a separate embodiment. While each claim may stand on its own as a separate embodiment, it is to be noted that—although a dependent claim may refer in the claims to a specific combination with one or more other claims—other embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

### LIST OF REFERENCE SIGNS

1.1, 1.2 Wearable device/Wearable patch 2 Chest
3 Upper arm
4 ECG signal
5 Pulse signal
6 Electrical impulse signal
7 Coordination device
8 Rectangular electrical impulse
B Human body

1. Method for synchronization of a multitude of wearable devices, wherein each wearable device is attached to a body (B) of a living organism, wherein a software application running on a coordination device sends an application time information to each wearable device such as to allow the wearable devices to synchronize.

2. Method according to claim 1, wherein at least one of the wearable devices is a wearable patch for monitoring at least one body signal, a pulse signal of the body (B), and/or an ECG signal of the body (B).

3. Method according to claim 1, wherein the coordination device is a mobile phone and/or a tablet computer and/or a laptop and/or a computer and/or a Bluetooth hub and/or a router and/or any type of hardware device.

4. Method according to claim 1, wherein the application time information is sent to the wearable devices via a wireless network using a wireless protocol such as Bluetooth and/or Zigbee and/or WiFi and/or GSM.

5. Method according to claim 1, wherein the application time information corresponds to an internal time of the coordination device, wherein the internal time is a network time, a time provided by a mobile carrier network, or a time set by the user.

6. Method according to claim 1, wherein each wearable device stores the application time information in a wearable device processor comprised in each wearable device, and starts a counter for maintaining a wearable device time in line with the application time information and in line with the internal time of the coordination device.

7. Method according to claim 6, wherein, in each particular wearable device, the counter of that particular wearable device uses a sampling rate of at least one sensor comprised in that particular wearable device for counting, wherein the sensor is a pulse sensor.

8. Method according to claim 1, wherein the multitude of wearable devices comprises at least two wearable devices, wherein the wearable devices are essentially identical.

9. Method according to claim 1, wherein the method comprises a first calibration phase, wherein the first calibration phase comprises the steps:
each wearable device measures a first characteristic point of a body signal, wherein the body signal is an ECG signal of the living organism, wherein the characteristic point is an R-peak of a QRS complex of the ECG signal;
each wearable device determines time-stamps of subsequent characteristic points, being of a same type as the first characteristic point, based on its particular counter;
each wearable device sends the time-stamps to the coordination device.

10. Method according to claim 9, wherein the first calibration phase is followed by a second calibration phase, wherein the second calibration phase comprises:
a time offset calculation step during which the coordination device calculates a particular time offset value for at least one of the wearable devices based on the time-stamps received from the wearable devices,
an offset transmission step during which the coordination device transmits at least to each of those wearable devices for which the particular time offset value does not equal “0” and/or to each of those wearable devices for which a time offset value has been calculated its particular time offset value;
a time adjustment step during which each wearable device that received its particular time offset value from the coordination device uses this particular time offset value to offset its particular wearable device time.

11. Method according to claim 10, wherein during the time offset calculation step, the coordination device calculates a mean time difference based on the time stamps for at least one of the wearable devices and saves this mean time difference as the particular time offset value.

12. Method according to claim 10, wherein
the time offset calculation step comprises the sub-steps:
the coordination device chooses one wearable device as reference wearable device;
the coordination device calculates time differences between the time-stamps of the reference wearable device and corresponding time-stamps of each other wearable device such as to obtain a multitude of time differences for each pair of the reference wearable device and any of the other wearable devices;
for each pair of the reference wearable device and any of the other wearable devices, the coordination device calculates a mean time difference based on the respective multitude of time differences;
for each other wearable device, the coordination device saves the determined mean time difference as the particular time offset value, and
the offset transmission step comprises the sub-step:
the coordination device transmits to each of the other wearable devices its particular time offset value.

13. Method according to claim 1, wherein the wearable devices only transmit information to the coordination device when the signal quality is good enough.

14. Method according to claim 9, wherein the characteristic point is a peak of pulse or a foot of pulse and/or that not all wearable devices use the same type of characteristic point, wherein each wearable device uses a different type of characteristic point.

15. Method according to claim 9, wherein a characteristic point is considered to be noisy if an RMS of an accelerometer value around an R-peak exceeds a certain threshold.

16. Method according to claim 1, wherein at least one wearable device periodically sends a distinct shape electrical impulse through the body to the other wearable devices so that the other wearable devices can use that distinct shape electrical impulse for synchronization.

17. Method according to claim 16, wherein the distinct shape electrical impulse has an amplitude that is higher than a typical amplitude of an ECG signal, wherein the amplitude of the distinct shape electrical impulse is higher than 10 mV.

18. Method according to claim 16, wherein the distinct shape electrical impulse is generated by at least one wearable device when this at least one wearable device measures a characteristic point on a particular body signal.

19. Method according to claim 1, wherein a moving time window and/or an instantaneous frequency waveform is used for synchronization in heavy noise.

20. Method according to claim 1, wherein at least one timing value is calculated once all patches are synchronized.
